(19) United States
(12) Patent Application Publication
(10) Pub. No.: US 2026/0077019 A1
(43) Pub. Date: Mar. 19, 2026

(54) TRIZEPATIDE AND OTHER GLP1RA AS A NOVEL TREATMENT FOR PATIENTS WITH LIPODYSTROPHY

(71) Applicant: Rutgers, The State University of New Jersey, New Brunswick, NJ (US)

(72) Inventors: Christopher Buettner, Mamaroneck, NY (US); Svetlana Ten, Staten Island, NY (US)

(73) Assignee: Rutgers, The State University of New Jersey, New Brunswick, NJ (US)

(21) Appl. No.: 19/332,597

(22) Filed: Sep. 18, 2025

Related U.S. Application Data
(60) Provisional application No. 63/696,068, filed on Sep. 18, 2024.

(51) Int. Cl. A61K 38/26; A61K 38/28; A61P 3/00

(52) U.S. Cl. CPC A61K 38/26; A61K 38/28; A61P 3/00

(57) ABSTRACT

The present disclosure provides methods of treating congenital generalized lipodystrophy using tirzepatide. The disclosure also provides methods for treating a disease in a patient characterized by leptin and leptin signaling deficiency.

### CROSS REFERENCE TO RELATED APPLICATIONS

This application is entitled to priority pursuant to 35 U.S.C. § 119(e) to U.S. Provisional Application No. 63/696,068, filed on Sep. 18, 2024. The content of the application is incorporated herein by reference in its entirety

### TECHNICAL FIELD

The present disclosure relates to methods of treating congenital generalized lipodystrophy and other diseases using a GLP-1 receptor agonist.

### BACKGROUND

Congenital generalized lipodystrophy (CGL) is a rare autosomal recessive disorder which is characterized by the near total loss of adipose tissue. A hallmark of CGL is severe insulin resistance resulting in lipoatrophic diabetes, hypertriglyceridemia, and hepatic steatosis. The absence of adipose tissue results in leptin deficiency which is a distinguishing feature of CGL from partial lipodystrophy where leptin levels are commonly not low and insulin resistance and metabolic disease is typically less severe. Recombinant leptin (Metreleptin) is the only FDA specific therapy approved for patients with CGL which improves insulin resistance, glucose homeostasis, hypertriglyceridemia and hepatic steatosis. Metreleptin is expensive, requires daily injections that are painful due to the absence of subcutaneous adipose tissue and in some cases loses efficacy due to the formation of neutralizing antibodies. In addition to leptin therapy, insulin sensitizers such as thiazolidinediones (TZDs) or insulin itself have been utilized to improve glycemic control and dyslipidemia, albeit with varying degrees of success.

Glucagon-like peptide-1 receptor agonist (GLP-1) treatment is used to improve insulin sensitivity in common forms of type 2 diabetes but has not been used in patients with CGL. Tirzepatide (Mounjaro) is a co-agonist of the GLP-1 and glucose-dependent insulinotropic polypeptide (GIP) receptors that is given weekly, leading to improved blood glucose control, insulin sensitivity and weight loss. Herein, the treatment of patients with CGL and severe insulin resistance with Tirzepatide is described.

### SUMMARY

In one aspect, the present disclosure provides a method of treating generalized lipodystrophy in a subject by administering a therapeutically effective amount of a GLP-1 receptor agonist to the subject.

In some embodiments, the subject has a 1-acylglycerol-3-phosphate O-acyltransferase 2 (AGPAT2) gene mutation resulting in CGL type 1 (CGL1). In some embodiments, the subject has a Berardinelli-Seip congenital lipodystrophy type 2 (BSCL2) gene mutation resulting in CGL type 2 (CGL2). In some embodiments, the subject has a caveolin-1 protein (CAV1) gene mutation resulting in CGL type 3 (CGL3). In some embodiments, the subject has a polymerase I and transcript release factor (PTRF) gene mutation resulting in CGL type 4 (CGL4).

In some embodiments, the subject has a deficiency in leptin. In some embodiments, the subject does not have adipose tissue. In some embodiments, the subject has severe insulin resistance resulting from CGL. In some embodiment, subjects need to be treated with insulin. In some embodiments, the subject exhibits signs of insulin resistance. In some embodiments, the subject is diabetic. In some embodiments, the subject is not diabetic.

For CGL subjects with diabetes, the GLP-1 receptor agonist can be co-administered with insulin. The glucose levels of a subject being co-administered the GLP-1 receptor agonist and insulin may return to normal levels, in which the co-administration of insulin is no longer required. Therefore, in some embodiments, the GLP-1 receptor agonist is administered to a subject without insulin. The GLP-1 receptor agonist can also be administered in different doses as described herein.

In another aspect, the disclosure provides a method of treating a disease in a subject characterized by leptin and leptin signaling deficiency by administering a therapeutically effective amount of a GLP-1 receptor agonist to the subject.

### BRIEF DESCRIPTION OF THE DRAWINGS

FIG. 1 shows the normalization of glucose levels after the patient received a weekly dose of 15 mg of tirzepatide. The dose-dependent effect is evident as indicated by the inability to control glucose levels at a dosage of 7.5 mg.

FIG. 2 shows the average glucose and glucose management indicator (GMI) after 1 week of administering 7.5 mg Tirzepitide and after 3 weeks of 15 mg Tirzepatide.

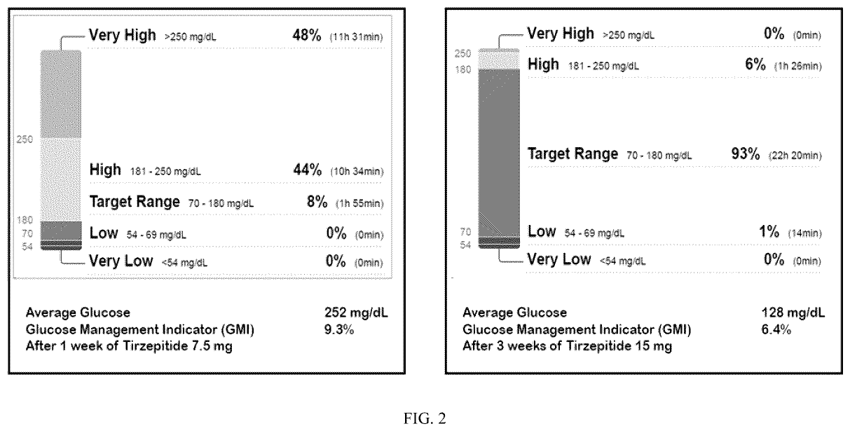

FIG. 3 shows the normalization of glucose levels on Tirzeitide 15 mg monotherapy off Insulin and off Metroleptin.

FIG. 4 shows the average glucose and glucose management indicator (GMI) after 1 week of Tirzepitide of 7.5 mg and 15 units of Lispro daily and after 1 week of 15 mg of Tirzepatide monotherapy.

### DETAILED DESCRIPTION

The present disclosure relates to a method of treating lipodystrophy in a subject by administering a therapeutically effective amount of a GLP-1 receptor agonist to the subject. The disclosure also provides methods of treating a disease in a subject characterized by leptin and leptin signaling deficiency by administering a therapeutically effective amount of a GLP-1 receptor agonist to the subject.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” as used herein refers to the referenced numeric indication plus or minus 10% of that referenced numeric indication.

A “therapeutically effective amount” of tirzepatide may vary according to factors such as the disease state, age, sex, and weight of the individual, and the ability of tirzepatide to elicit a desired response in the individual. A therapeutically effective amount is also one in which any toxic or detrimental effects of tirzepatide are outweighed by the therapeutically beneficial effects. The term “therapeutically effective amount” refers to an amount of tirzepatide or composition comprising the same, which is effective to treat a disease or disorder in a subject (e.g., a patient).

“Treat,” “Treatment,” or “treating,” as used herein, includes any desirable effect on the symptoms or pathology of a disease or condition associated with glucagon-like peptide-1 receptor (GLP-1) inactivity and glucose-dependent insulinotropic polypeptide (GIP), or a risk of developing such a condition, and may include even minimal changes or improvements in one or more measurable markers of the disease or condition being treated. “Treatment” or “treating” does not necessarily indicate complete eradication or cure of the disease or condition, or associated symptoms thereof.

The term “GLP-1 receptor agonist” as used herein refers to any drug that interacts with a GLP-1 receptor. The GLP-1 receptor agonist may be a mono-agonist, a dual-agonist, a triple-agonist, a poly-agonist, or a co-agonist. In some embodiments, the GLP-1 receptor agonist is an agonist of glucagon receptors, amylin receptors, selective peptide tyrosine tyrosine (PYY) receptors, glucose-dependent insulinotropic polypeptide (GIP) receptors, or any combination thereof. In a preferred embodiment, the GLP-1 receptor agonist is a dual agonist of the GLP-1 and glucose-dependent insulinotropic polypeptide (GIP) receptors. In some embodiments, the GLP-1 receptor agonist is selected from the group consisting of tirzepatide, semaglutide and retatrutide. In a preferred embodiment, the GLP-1 receptor agonist is tirzepatide.

The “subject” receiving this treatment is intended to include human and non-human animals. Non-human animals include all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. In a preferred embodiment, the “subject” is a human patient suffering from CGL.

### Method of Treating Congenital Generalized Lipodystrophy

In one aspect, the present disclosure provides a method of treating generalized lipodystrophy in a subject by administering a therapeutically effective amount of a GLP-1 receptor agonist to the subject. In some embodiments, the GLP-1 receptor agonist is selected from the group consisting of tirzepatide, semaglutide and retatrutide. In a preferred embodiment, the GLP-1 receptor agonist is tirzepatide.

In some embodiments, the subject has a 1-acylglycerol-3-phosphate O-acyltransferase 2 (AGPAT2) gene mutation resulting in CGL1. In some embodiments, the AGPAT2 gene mutation is selected from the group consisting of: c.IVS4-2A>G, rs104894093 (ARG68TER), rs116807569 (c.589-2A>G), rs387906355 (1-BP INS 377T), rs104894100 (LEU228PRO), rs387906356 (3-BP DEL, 418TTC), rs121908925 (LYS215TER), rs606231168 (IVS3 A-G, -1), rs121908926 (PHE189TER), rs1255380257 (GLU229TER), and rs748157664 (GLU172LYS).

In some embodiments, the subject has a Berardinelli-Seip congenital lipodystrophy type 2 protein (BSCL2) gene mutation resulting in CGL2. In some embodiments, the BSCL2 gene mutation is selected from the group consisting of: rs786205068 (2-BP DEL/3-BP INS, NT536), rs786205069 (2-BP INS, 645AA), rs786205070 (2-BP DEL, 659GT), rs587777608 (5-BP DEL, NT659), rs786205071, (1-BP INS, 669A), rs137852970 (ARG138TER), rs1945400235 (IVS4, G-A, +1), rs13785297 (ALA212PRO), rs758843908 (1-BP DEL, 980C), rs786205072 (IVS6, G-A, +5), and rs786205073 (IVS6, C-G, -3).

In some embodiments, the subject has a caveolin-1 protein (CAV1) gene mutation resulting in CGL3. In some embodiments, the CAV1 gene mutation is selected from the group consisting of: rs121434501 (GLU38TER) and 2-BP DEL, NT237.

In some embodiments, the subject has a polymerase I and transcript release factor (PTRF) gene mutation resulting in CGL4. In some embodiments, the PTRF gene mutation is selected from the group consisting of: 696insC and 525delG.

In some embodiments, the subject has a blood plasma concentration of leptin of 0.5 ng/mL or less. In some embodiments, the subject has a blood plasma concentration of leptin of 0.4 ng/mL or less. In some embodiments, the subject has a blood plasma concentration of leptin of 0.3 ng/mL or less. In some embodiments, the subject has a blood plasma concentration of leptin of 0.2 ng/mL or less. In some embodiments, the subject has a blood plasma concentration of leptin of 0.1 ng/mL or less.

Although, patients with CGL are not obese, the absence of adipose tissue and the resulting leptin deficiency leads to diabetes due to extreme insulin resistance. In some embodiments, the patient has less than 5% adipose tissue as measured by dual-energy X-ray absorptiometry (DXA) scans. In some embodiments, the patient has less than 4% adipose tissue. In some embodiments, the patient has less than 3% adipose tissue. In some embodiments, the patient has less than 2% adipose tissue. In some embodiments, the patient has less than 1% adipose tissue. In some embodiments, the patient has less than 0.1% adipose tissue. In some embodiments, the subject does not have adipose tissue. In some embodiments, the subject has severe insulin resistance resulting from CGL. In some embodiment, subjects need to be treated with insulin. In some embodiments, the subject exhibits signs of insulin resistance. In some embodiments, the subject is diabetic. In some embodiments, the subject is not diabetic.

For CGL subjects with diabetes, the GLP-1 receptor agonist can be co-administered with insulin. The glucose levels of a subject being co-administered the GLP-1 receptor agonist and insulin may return to normal levels, in which the co-administration of insulin is no longer required. Therefore, in some embodiments, the GLP-1 receptor agonist is administered to a subject without insulin.

The GLP-1 receptor agonist can be administered in different doses. In some embodiments, the therapeutically effective dose to be administered is about 2.5 mg per 0.5 mL. In some embodiments, the therapeutically effective dose to be administered is about 5 mg per 0.5 mL. In some embodiments, the therapeutically effective dose to be administered is about 7.5 mg per 0.5 mL. In some embodiments, the therapeutically effective dose to be administered is about 10 mg per 0.5 mL. In some embodiments, the therapeutically effective dose to be administered is about 12.5 mg per 0.5 mL. In some embodiments, the therapeutically effective dose to be administered is about 15 mg per 0.5 mL.

### Method of Treating a Disease Characterized by Leptin and Leptin Signaling Deficiency

As discussed above, lipodystrophy is characterized by deficiency of adipose tissue, which can lead to leptin deficiency. Further, in the present disclosure, the GLP-1 receptor agonist may also be administered to subjects with disease states that are characterized by leptin and leptin signaling deficiency. In some embodiments, lipodystrophy and the disease states characterized by leptin and leptin signaling deficiency have at least one common symptom, i.e., low circulating levels of leptin.

In another aspect, the disclosure provides a method of treating a disease in a subject characterized by leptin and leptin signaling deficiency by administering a therapeutically effective amount of a GLP-1 receptor agonist to the subject. In some embodiments, the GLP-1 receptor agonist is selected from the group consisting of tirzepatide, semaglutide and retatrutide. In a preferred embodiment, the GLP-1 receptor agonist is tirzepatide.

The disease can be selected from, but not limited to CGL, familial partial lipodystrophy, acquired generalized lipodystrophy (AGL), acquired partial lipodystrophy, high active antiretroviral therapy-induced lipodystrophy, localized lipodystrophy, congenital leptin deficiency, acquired hypoleptinemia. In some embodiments, the acquired generalized lipodystrophy is Lawrence syndrome. A subject suffering from an acquired form of generalized lipodystrophy (i.e. Lawrence syndrome) may have etiologies including previous viral infection or autoimmunity associated with panniculitis.

The subject may also have a leptin signaling deficiency due to a mutation in the leptin gene (LEP) or leptin receptor gene (LEPR). In some embodiments, the subject has a LEP gene mutation selected from the group consisting of: rs104894023 (ARG105TRP), rs200575914 (GLY59SER), and 1-BP DEL, FS147TER. In some embodiments, the subject has a LEPR gene mutation selected from the group consisting of: rs1474810899 (IVS16DS, G-A, +1), rs1557670950 (TYR155TER).

### EXAMPLES

### Example 1

A 23-year-old African American male patient was diagnosed with diabetes type 1 at 9 years of age. Treatment with high doses of insulin (more than 300 units/daily) with addition of metformin was unsuccessful in controlling his blood glucose. At 11-years-old, the patient was diagnosed with CGL and was noted to have significant acanthosis nigricans consistent with severe insulin resistance. The patient's BMI was 23.2 kg/m² and blood pressure was normal.

The patient's leptin level were notably low at 0.5 ng/mL (Normal range: 1.4-16.5), accompanied by elevated triglycerides of 238 mg/dL, ALT at 36 U/L, AST at 34 U/L, and a C-peptide level of 7.06 ng/mL. Hemoglobin A1c was substantially elevated at 14.7%, while Islet cell antibodies tested negative (Table 1). An MRI revealed generalized muscle hypertrophy with significantly reduced subcutaneous and intra-abdominal fat, periarticular and intramedullary lytic bone lesions, and hepatomegaly. The CGL diagnosis was confirmed by the identification of a homozygous AGPAT2 (1-acylglycerol-3-phosphate O-acyltransferase 2) gene mutation (c.IVS4-2A>G).

Treatment commenced with a daily dose of 2.5 mg of Metreleptin at the age of 14, leading to the cessation of insulin therapy within one month. Remarkably, without the need for any additional medications, the patient's glucose levels normalized. Furthermore, after 6 months of Metreleptin therapy, the patient's HbA1c improved from 11% to 5.9%. Concurrently, the patient's triglycerides, ALT, and AST levels normalized as well (Table 1). Neutralizing antibodies, assessed after 2 years of therapy, were within normal limits.

TABLE 1
Summary of the test results during treatment.

| | | Before | | | |
|---|---|---|---|---|---|
| | | Leptin | | | At 23 years |
| | Insulin 300 | treatment | 6 months | At 22 years | After |
| | units daily at | at 14 | after | off | Tirzepatide |
| | 12 years | years | Leptin | medications | 2.5-7.5 mg |
| Glucose fasting 65-99 mg/dL | 328 | 235 | 119 | 624 | 198 |
| C-peptide 0.8-3.1 ng/ml | 7.06 | 0.92 | 4.08 | 2.05 | 4.56 |
| HbA1c <5.6% | 14.7 | 11 | 5.9 | >14 | 13.9 |
| Leptin 1.4-16.5 ng/ml | 0.5 | 1.3 | 1.3 | 0.9 | 0.5 |
| AST 12-32 U/L | 34 | 19 | 28 | 14 | 21 |
| ALT 6-19 U/L | 36 | 19 | 25 | 21 | 19 |
| Cholesterol 125-170 mg/dL | 110 | 111 | 91 | 139 | 135 |
| HDL 35-75 mg/dL | 36 | 41 | 41 | 51 | 54 |
| LDL <110 mg/dL | 25 | 29 | 43 | 60 | 66 |
| Triglycerides 36-135 mg/dL | 238 | 204 | 36 | 212 | 66 |
| Microalbumin/creatinine | 5 | 6 | 17 | 300 | |
| <30 mcg/mg creat | | | | | |

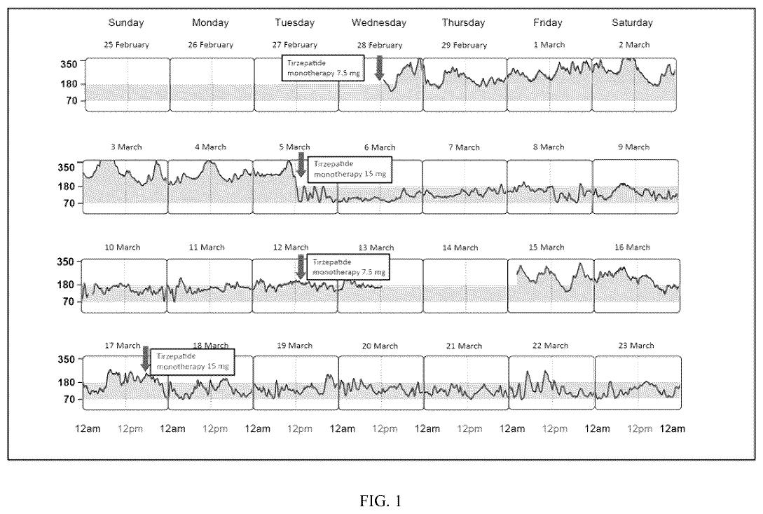

At 16 years of age, the patient discontinued all treatments, resulting in the increase in HbA1c to 14%. Subsequently, the patient was hospitalized, and insulin requirement surged again to 300 units/day (4 units/kg/day) prompting the reinitiation of Metreleptin therapy. However, six months later, the patient ceased all therapy once more, including both insulin and Metreleptin. The patient persisted in refusing treatment and follow-up until returning at 23 years of age. At this juncture, Tirzepatide treatment was initiated, without the use of Metreleptin or insulin. Despite increasing doses of Tirzepatide (from 2.5 mg to 7.5 mg), glucose levels remained elevated. However, upon increasing the dosage to 15 mg weekly, glucose levels normalized, demonstrating a clear dose-dependent effect (FIG. 1). Notably, the patient tolerated the treatment well, experiencing no adverse side effects such as nausea, diarrhea, constipation, or abdominal pain, even though the tirzepatide was rapidly increased. Tirzepatide was rapidly titrated as follows: 2.5 mg weekly in the first week, 5 mg in the second week, 7.5 mg in the third week, and 15 mg in the fourth week.

### Example 2

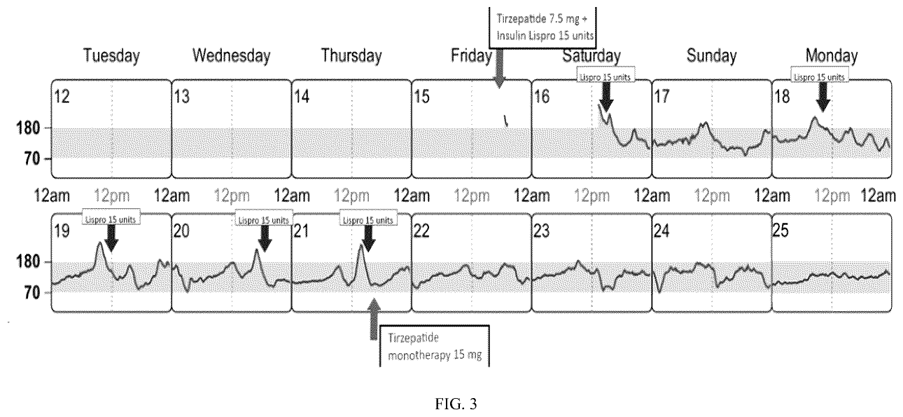

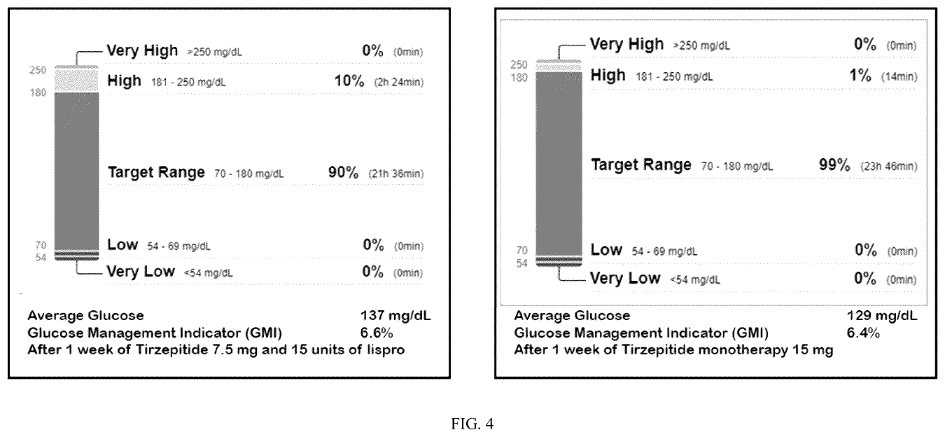

A 64-year-old African American woman was diagnosed with CGL and Diabetes at 39-years-old, confirmed by homozygous AGPAT2 gene mutation. The patient's lipodystrophy was complicated by severe insulin resistance, diabetes, diabetic nephropathy, elevated triglycerides, low HDL cholesterol, fatty liver, bone lesions, polycystic ovary syndrome (PCOS), and clitoromegaly. The patient was treated with Metreleptin 5 mg daily from 2001, Levomir 20 units daily, Humalog 15 units before food, Enalapril 20 mg daily and Simvastatin 20 mg daily. The patient's HbA1c was 6.4%, indicating good glycemic control, but the patient required multiple injections per day of insulin and 1 injection daily of Metreleptin, a total of 5 injections per day. To reduce the number of daily injections the patient opted to start a trial of weekly Tirzepitide. Metreleptin and insulin detemir were discontinued while lispro insulin was retained for use as needed for glucose levels above 180 mg/dL. Tirzepatide was rapidly titrated as follows: 2.5 mg weekly in the first week, 5 mg in the second week, 7.5 mg in the third week, and 15 mg in the fourth week. Insulin treatment was discontinued after the 3rd week when Tirzepitide dose was increased to 15 mg per week. Within one week of Tirzepatide 15 mg per week monotherapy the patient's glucose levels normalized as per continuous glucose monitoring (CGM) tracings (FIG. 3). After 1 week of 7.5 mg of Tirzepatide and 15 units of lispro insulin/day, the average glucose was 137 mg/dL, the GMI was 6.6% and 90% of glucose readings were within the target range. After 1 week of 15 mg of Tirzepatide monotherapy the average glucose was 129 mg/dL. GMI was 6.4% and target in range glucose was 99% (FIG. 4).

The patient developed diarrhea after 2 days of increasing the dose of Tirzepatide to 15 mg. Despite the diarrhea resolving after 2 days, the patient reverted back to her previous treatment regimen with Metreleptin and Insulin and discontinued Tirzepatide. The patient's hunger was similarly controlled by Metreleptin and Tirzepatide.

1. A method of treating generalized lipodystrophy in a patient in need thereof, the method comprising administering a therapeutically effective amount of a GLP-1 receptor agonist to the patient.

2. The method of claim 1, wherein the GLP-1 receptor agonist is selected from the group consisting of tirzepatide, semaglutide and retatrutide.

3. The method of claim 1, wherein the patient has a 1-acylglycerol-3-phosphate O-acyltransferase 2 (AGPAT2) gene mutation, a Berardinelli-Seip congenital lipodystrophy type 2 protein (BSCL2) gene mutation, a caveolin-1 protein (CAV1) gene mutation, or A polymerase I and transcript release factor (PTRF) gene mutation.

4. The method of claim 3, wherein the AGPAT2 gene mutation selected from the group consisting of: c.IVS4-2A>G, rs104894093 (ARG68TER), rs116807569 (c.589-2A>G), rs387906355 (1-BP INS 377T), rs104894100 (LEU228PRO), rs387906356 (3-BP DEL, 418TTC), rs121908925 (LYS215TER), rs606231168 (IVS3 A-G, -1), rs121908926 (PHE189TER), rs1255380257 (GLU229TER), and rs748157664 (GLU172LYS).

5. The method of claim 3, wherein the BSCL2 gene mutation selected from the group consisting of: rs786205068 (2-BP DEL3-BP INS, NT536), rs786205069 (2-BP INS, 645AA), rs786205070 (2-BP DEL, 659GT), rs587777608 (5-BP DEL, NT659), rs786205071, (1-BP INS, 669A), rs137852970 (ARG138TER), rs1945400235 (IVS4, G-A, +1), rs13785297 (ALA212PRO), rs758843908 (1-BP DEL, 980C), rs786205072 (IVS6, G-A, +5), and rs786205073 (IVS6, C-G, -3).

6. The method of claim 3, wherein the CAV1 gene mutation selected from the group consisting of: rs121434501 (GLU38TER) and 2-BP DEL, NT237.

7. The method of claim 3, wherein the PTRF gene mutation selected from the group consisting of: 696insC and 525delG.

8. The method of claim 1, wherein the patient does not have adipose tissue.

9. The method of claim 1, wherein the patient has a deficiency in leptin.

10. The method of claim 1, wherein the patient has a blood plasma concentration of leptin of 0.5 ng/mL or less.

11. The method of claim 1, wherein the patient is diabetic or not diabetic.

12. The method of claim 1, wherein the patient has severe insulin resistance resulting from generalized lipodystrophy.

13. The method of claim 12, wherein the GLP-1 receptor agonist is co-administered with insulin, wherein after the patient's glucose levels normalize, the GLP-1 receptor agonist is administered without insulin.

14. The method of any one of claim 1, wherein the therapeutically effective dose of the GLP-1 receptor to be administered is about 2.5 mg per 0.5 mL, about 5 mg per 0.5 mL, about 7.5 mg per 0.5 mL, about 10 mg per 0.5 mL, about 12.5 mg per 0.5 mL, or about 15 mg per 0.5 mL.

15. A method of treating a disease in a patient characterized by leptin and leptin signaling deficiency, the method comprising administering a therapeutically effective amount of a GLP-1 receptor agonist to the patient.

16. The method of claim 15, wherein the disease is selected from the group consisting of: CGL, CGL1, CGL2, CGL3, CGL4, familial partial lipodystrophy, acquired generalized lipodystrophy (AGL), acquired partial lipodystrophy, high active antiretroviral therapy-induced lipodystrophy, localized lipodystrophy, congenital leptin deficiency, and acquired hypoleptinemia.

17. The method of claim 15, wherein the GLP-1 receptor agonist is selected from the group consisting of tirzepatide, semaglutide and retatrutide.

18. The method of claim 15, wherein the leptin and leptin signaling deficiency results from mutations in the leptin (LEP) gene or leptin receptor (LEPR) gene.

19. The method of claim 18, wherein the patient has a LEP gene mutation selected from the group consisting of: rs104894023 (ARG105TRP), rs200575914 (GLY59SER), and 1-BP DEL, FS147TER.

20. The method of claim 18, wherein the patient has a LEPR gene mutation selected from the group consisting of: rs1474810899 (IVS16DS, G-A, +1), rs1557670950 (TYR155TER).
